(19) United States
(12) Patent Application Publication
(10) Pub. No.: US 2010/0317562 A1
(43) Pub. Date: Dec. 16, 2010

(54) Dietary Compositions

(76) Inventors: Lynn Paolella, Framingham, MA (US); David Paolella, Framingham, MA (US)

Correspondence Address: FISH & RICHARDSON P.C. (BO), P.O. BOX 1022, MINNEAPOLIS, MN, 55440-1022, US

(21) Appl. No.: 12/532,805

(22) PCT Filed: Mar. 24, 2008
(86) PCT No.: PCT/US08/58005

§ 371 Date: Jul. 29, 2010

Related U.S. Application Data
(60) Provisional application No. 60/896,837, filed on Mar. 23, 2007.

(51) Int. Cl. A61K 38/02; A61K 31/4172; A61P 3/00

(52) U.S. Cl. 514/1.1; 514/400

(57) ABSTRACT

Compositions that include medical food emulsions are provided. The compositions can include a low pH solvent and amino acids wherein the amino acids are exclusive of at least one naturally occurring amino acid. Also provided are methods of making compositions that include medical food emulsions. Such medical food emulsions are useful in the nutritional support of individuals with inborn errors of protein metabolism.

This application claims priority to U.S. Application Ser. No. 60/896,837, filed on Mar. 23, 2007. For the purpose of any U.S. patent that may issue based on the present application, U.S. Application Ser. No. 60/896,837 is hereby incorporated by reference herein in its entirety.

### TECHNICAL FIELD

This invention relates to food products and methods for their manufacture, and more particularly to food products formulated for people who have an inborn error in protein metabolism.

### BACKGROUND

Providing proper nutrition to individuals with inborn errors of protein metabolism (IEPM) is extremely difficult, if not impossible, using a natural diet. IEPM patients detected in the course of newborn screening are immediately placed on a severely restricted protein diet and given amino acid supplements. Frequent blood monitoring is required to confirm dietary compliance and to validate the nutritional quality of the prescribed, protein-restricted diet. Currently, affected individuals consume few fruits, and vegetables, particularly vegetables known to be protein-rich, such as spinach, potatoes, and corn. Their diet is also limited in pure carbohydrates and lipid products and is essentially free of all meats, fish, dairy products, and legumes. This restricted diet is supplemented with elemental formulas comprised of carbohydrate, fats, and free amino acids meant to be administered as a hydrated drink. The current state of the art leaves little food choices and an amino acid drink to be consumed several times throughout the day for the entire lifetime of the IEPM patient. While adequate for infants and toddlers, dietary compliance can be difficult for individuals capable of eating solid foods. A combination of poor taste and social stigma associated with consuming drinks resembling baby formulas conspire against patients and their families from about age five onwards.

### SUMMARY

The present invention is based, in part, on our discovery of dietary compositions, including medical food emulsions that include or that consist of (a) a low pH solvent; and (b) a blend of amino acids suitable for administration to a patient having an inborn error in protein metabolism. The low pH solvent can include about 5% to 60% (e.g., about 5, 10, 15, 20, 25, 30, 35, 40, 45, 50, 55 or 60%) of the emulsion, by weight or by volume, and the blend of amino acids can comprise about 10% to 60% (e.g., about 10, 15, 20, 25, 30, 35, 40, 45, 50, 55 or 60%) of the emulsion by weight. The amino acid blend excludes at least one naturally occurring amino acid residue to accommodate a patient's intolerance of that amino acid residue. The pH of the low pH solvent can vary, as can the pH of the total food emulsion. For example, the low pH solvent can have a pH of about 0.5 to 5.0 (e.g. of about 1.0 to about 3.5; about 1.5 to 3.0; about 2.0 to 3.0; about 2.0 to 4.0; about 3.0 to 5.0). Specific pH levels of the low pH solvent can be about 0.5, 0.8, 1.0, 1.2, 1.5, 1.7, 2.0, 2.2, 2.5, 2.7, 3.0, 3.3, 3.5, 3.6, 3.7, 3.8, 3.9, 4.0, 4.2, 4.5, 4.8, or 5.0. The food emulsion at one or more stages of production, including the stage that constitutes the final product can have a pH of about 2.0 to about 6.0 (e.g., about 2.0 to 3.0; about 2.0 to 4.0; about 2.0 to 5.0; about 2.5 to 3.0; about 3.0 to 3.5; about 3.5 to 4.0; about 4.0 to 4.5 or about 4.5 to 5.0). Specific pH levels of the food emulsion are about 2.2, 2.5, 2.7, 3.0, 3.3, 3.5, 3.6, 3.7, 3.8, 3.9, 4.0, 4.2, 4.5, 4.8, 5.0, or 5.5.

The low pH solvent can be an organic acid (e.g., an acetic acid, citric acid, lactic acid, adipic acid, malic acid, tartaric acid, or other non-toxic acid suitable for the preparation of food, or a combination of two or more such acids). The organic acid can also be contained within, or may include, a vinegar.

The amino acids in the various amino acid blends can be present in a variety of forms. For example, the amino acids can be present as a mixture of single amino acid residues. Alternatively, or in addition, some of the amino acids can be joined to one another to form di-peptides, tri-peptides, or peptides of other lengths. In some embodiments, the peptides can be fragments of full-length naturally occurring or synthetic proteins. The amino acid residue excluded from the amino acid blend can be leucine, isoleucine, valine, or phenylalanine. One of ordinary skill in the art will be guided by the information contained herein and by information available to the public regarding amino acid blends suitable for administration to a patient having an IEPM. We tend to use the terms “blend”, “formulation” and “mixture” interchangeably.

Any of the medical food emulsions described above can further include one or more of the following: (a) a carbohydrate (e.g., a disaccharide, such as sucrose, or a starch); (b) a lipid (e.g., a vegetable oil); (c) a vitamin; and (d) a mineral. Where a starch is included it can be, for example, a non-modified starch, a modified starch, an instant starch, a cook-up starch, or any combination thereof. The starch can also be a corn, waxy cornstarch, potato, rice, tapioca, or wheat starch or any combination thereof. Suitable vegetable oils include soybean oil, sunflower oil, canola oil, low erucic rapeseed oil, cottonseed oil, corn oil, olive oil, or any combination thereof.

Any of the medical food emulsions described herein can further include an antioxidant, a coloring agent, a stabilizer, a preservative, a flavoring agent, or any combination of these agents.

More specifically, the medical food emulsion can include, or can consist of, balsamic vinegar, water, mustard, honey, oil, an emulsifier, and a blend of amino acids suitable for administration to a patient having an inborn error in protein metabolism.

Other medical food emulsions can include a non-dairy creamer, lemon juice, a vitamin and/or mineral, a seasoning and/or spice, a mayonnaise or comparable emulsion, and a blend of amino acids suitable for administration to a patient having an inborn error in protein metabolism.

Other medical food emulsions can include an enchilada sauce, a vitamin and/or mineral, an organic acid, water, and a blend of amino acids suitable for administration to a patient having an inborn error in protein metabolism.

Other medical food emulsions can include balsamic vinegar, water, a vitamin and/or mineral, a seasoning and/or spice, an oil, and a blend of amino acids suitable for administration to a patient having an inborn error in protein metabolism.

Any of the emulsions can be formulated as a salad dressing or sauce.

The details of one or more embodiments of the invention are set forth in the accompanying drawings and the description below. Other features, objects, and advantages of the invention will be apparent from the description and drawings, and from the claims.

### DETAILED DESCRIPTION

Inborn errors of amino acid metabolism occur when there is a block in a pathway in a metabolic sequence. The block results in an accumulation of normal intermediary products in abnormally large amounts and also of products of usually little used metabolic pathways. In most instances this effect can be attributed to the accumulation of an intermediary product proximal to the block. The accumulated toxic effects of these intermediary metabolites can be treated by restricting the intake of the essential substance from which the toxic metabolite is derived. Sometimes, the block prevents the synthesis of an essential compound normally made distal to the block and therapy consists of specifically providing this metabolite.

Individual with certain inborn errors of amino acid or nitrogen metabolism can be treated with special diets that restrict one or more amino acids to the minimum amount essential for normal growth. The amount of the restricted amino acid provided by the diet must be sufficient to meet the metabolic requirements dependent on it, but it must not permit an excess accumulation in the body fluids of the amino acid or its derivatives, or of nitrogen.

Compositions:

The medical food emulsions of the present invention include amino acids. An amino acid is any molecule that contains both amine and carboxyl functional groups. In order to support metabolic processes, living organisms typically rely upon α-amino acids with the general formula NH₂CHRCOOH, i.e., amino acids in which the amino and carboxylate groups are both attached to the α-carbon. The various α-amino acids are distinguished by the particular side chain (R group) that is attached to the α-carbon. The R group can vary in size from a single hydrogen atom in glycine, through a methyl group in alanine, to a large heterocyclic group in tryptophan. Most amino acids occur in two possible optical isomers, called D and L. Although D amino acids can be found in nature, generally the L isomers are used to form proteins.

Amino acids are usually classified by the properties of the side chain into four groups. The side chain can make them behave like a weak acid, a weak base, a hydrophile or a hydrophobe. The phrase “branched-chain amino acids” or BCAA is sometimes used to refer to the amino acids having aliphatic side-chains that are non-linear, i.e., leucine, isoleucine and valine. Proline is the only proteinogenic amino acid whose side group links to the α-amino group, and thus is also the only proteinogenic amino acid containing a secondary amine at this position. Proline has sometimes been termed an imino acid.

As amino acids have both the active groups of an amine and a carboxylic acid they can be considered both acid and base (though their natural pH is usually influenced by the R group). At a certain pH known as the isoelectric point, the amine group gains a positive charge (is protonated) and the acid group a negative charge (is deprotonated). The exact value is specific to each different amino acid. This ion is known as a zwitterion. A zwitterion can be extracted from the solution as a white crystalline structure with a very high melting point, due to its dipolar nature. Near-neutral physiological pH allows most free amino acids to exist as zwitterions.

There are twenty standard amino acids used by cells in protein biosynthesis. These include: L-alanine, L-arginine, L-aspartic acid, L-cysteine, glycine, L-glutamic acid, L-glutamine, L-histidine, L-isoleucine, L-leucine, L-lysine, L-methionine, L-phenylalanine, L-proline, L-serine, L-threonine, L-tyrosine, L-tryptophan, and L-valine. These twenty amino acids can be synthesized from simpler molecules, but organisms differ in how many they are able to produce and essential amino acids must be obtained in their diet. Of the 20 standard proteinogenic amino acids, 10 are called essential amino acids because the human body cannot synthesize them from other compounds through chemical reactions, and they therefore must be obtained from food. Essential amino acids include isoleucine, leucine, lysine, methionine, phenylalanine, threonine, tryptophan, valine, and in certain cases, arginine and histidine. Non-essential amino acids include alanine, asparagine, aspartic acid, cysteine, glutamic acid, glutamine, glycine, praline, serine and tyrosine. Cysteine, tyrosine, histidine, arginine and taurine are considered as semiessential amino acids in children, because the metabolic pathways that synthesize these amino acids are not fully developed.

The amino acid formulations provided herein can be tailored for use in specific disorders of amino acid metabolism by removing the particular amino acid or acids that are detrimental to those individuals having such a disorder. For example, a formula that includes the standard amino acids, e.g., L-alanine, L-arginine, L-aspartic acid, L-cysteine, L-glycine, L-glutamic acid, L-glutamine, L-histidine, L-isoleucine, L-leucine, L-lysine, L-methionine, L-proline, L-serine, L-threonine, L-tyrosine, L-tryptophan, and L-valine, but lacks at least L-phenylalanine can be used to prepare medical food emulsions for individuals having disorders of phenylalanine metabolism e.g., phenylketeonuria. In another example, a formula that includes the standard amino acids, e.g., L-alanine, L-arginine, L-aspartic acid, L-cysteine, L-glutamic acid, L-glutamine, glycine, L-histidine, L-lysine, L-methionine, L-phenylalanine, L-proline, L-serine, L-threonine, L-tyrosine, and L-tryptophan, but lacks at least L-isoleucine, L-leucine, and L-valine can be used to prepare medical food emulsions for individuals having disorders of branched chain amino acid metabolism e.g., maple syrup urine disease (MSUD) or classic branched chain ketoaciduria (BCKA). Similarly, a formula that includes the standard amino acids e.g., L-alanine, L-arginine, L-aspartic acid, L-cysteine, glycine, L-glutamic acid, L-glutamine, L-histidine, L-isoleucine, L-lysine, L-methionine, L-phenylalanine, L-proline, L-serine, L-threonine, L-tyrosine, L-tryptophan, and L-valine, but lacks at least L-leucine can be used to prepare medical food emulsions for individuals having disorders of leucine metabolism, e.g., isovaleric academia. A formula that includes the standard amino acids e.g., L-alanine, L-arginine, L-aspartic acid, L-cysteine, glycine, L-glutamic acid, L-glutamine, L-histidine, L-isoleucine, L-leucine, L-lysine, L-methionine, L-proline, L-serine, L-threonine, L-tryptophan, and L-valine, but that lacks at least L-phenylalanine, L-tyrosine can be used to prepare medical food emulsions for individuals having disorders of tyrosine metabolism, e.g., tyrosinemia, types I and II, and alcaptonuria. A formula that includes the standard amino acids e.g., L-alanine, L-arginine, L-aspartic acid, L-cysteine, glycine, L-glutamic acid, L-glutamine, L-histidine, L-isoleucine, L-leucine, L-lysine, L-phenylalanine, L-proline, L-serine, L-threonine, L-tyrosine, L-tryptophan, and L-valine, but that lacks at least L-methionine, can be used to prepare medical food emulsions for individuals having disorders of cysteine metabolism, e.g., homocystinuria and hypermethioninemia. A formula that includes the standard amino acids, e.g., L-alanine, L-arginine, L-aspartic acid, L-cysteine, glycine, L-glutamic acid, L-glutamine, L-histidine, L-isoleucine, L-leucine, L-lysine, L-phenylalanine, L-proline, L-serine, L-threonine, L-tyrosine, and L-tryptophan, but lacks at least L-methionine, and L-valine can be used to prepare medical food emulsions for individuals having disorders of organic acid metabolism, e.g., methylmalonic acidemia and propionic acidemia. A formula that includes the standard amino acids, e.g., L-alanine, L-arginine, L-aspartic acid, L-cysteine, glycine, L-glutamic acid, L-histidine, L-isoleucine, L-leucine, L-methionine, L-phenylalanine, L-proline, L-serine, L-threonine, L-tyrosine, and L-valine, but lacks at least L-glutamine, L-lysine, L-tryptophan can be used to prepare medical food emulsions for individuals having disorders of glutaryl-CoA metabolism, e.g., glutaric aciduria type I. For example, a formula that includes the standard amino acids, e.g., L-arginine, L-cysteine, L-glutamine, L-histidine, L-isoleucine, L-leucine, L-lysine, L-methionine, L-phenylalanine, L-threonine, L-tyrosine, L-tryptophan, and L-valine, but lacks at least L-alanine, L-glutamic acid, L-proline, L-aspartic acid, L-serine, L-glycine, can be used to prepare medical food emulsions for individuals having urea cycle disorders e.g., N-acetyl glutamate synthase deficiency, carbamyl phosphate synthase deficiency, ornithine transcarbamylase deficiency, arginosuccinic acid synthase deficiency, arginosuccinate lyase deficiency, and arginase deficiency. The compositions can also include non-naturally occurring or non-standard amino acids, for example, selenocysteine or selenomethionine, where, of course, the non-naturally occurring or non-standard amino acids are not detrimental to those individuals having the specific disorder for which the compositions are intended.

The relative amounts of the individual amino acids included in specific formulations for particular disorders can vary and will depend upon the nature of the disorder for which the formulation is intended. Determination of relative amounts can be made by one of skill in the art. For example, for treating PKU, the amino acid blend of present composition can have 1-10% (w/w) L-alanine (e.g., 3-5% or about 4%); 5-15% (w/w) L-arginine (e.g., 6-10% or about 8%); 5-15% (w/w) L-aspartic acid (e.g., 6-10% or about 8%); 0.5-10% (w/w) L-cysteine (e.g., 2-4% or about 3%); 5-15% (w/w) glycine (e.g., 6-10% or about 8%); 1-10% (w/w) L-histidine (e.g., 3-5% or about 4%); 5-15% (w/w) L-isoleucine (e.g., 6-10% or about 8%); 5-15% (w/w) L-leucine (e.g., 9-12% or about 11%); 5-15% (w/w) L-lysine (e.g., 6-10% or about 8%); 0.5-10% (w/w) L-methionine (e.g., 1-3% or about 2%); 0% L-phenylalanine; 5-15% (w/w) L-proline 6-10% or about 7%); 1-10% (w/w) L-serine (e.g., 6-10% or about 5%); 5-15% (w/w) L-threonine (e.g., 6-10% or about 8%); 0.5-10% (w/w) L-tryptophan (e.g., 1-3% or about 2%); 5-15% (w/w) L-tyrosine (e.g., 9-12% or about 10%); 5-15% (w/w) L-valine (e.g., 6-10% or about 8%); 0.02-5% (w/w) L-carnitine (e.g., 0.06-0.1% or about 0.08%); and 0.02-5% (w/w) taurine (e.g., 0.2-0.5% or about 0.3%). An example of a suitable amino acid blend for PKU is shown in Table 1 in the Examples section.

For treating MSUD for example, the amino acid blend of present composition can have 5-15% (w/w) L-alanine (e.g., 9-12% or about 10%); 5-15% (w/w) L-arginine (e.g., 7-10% or about 9%); 5-15% (w/w) L-aspartic acid (e.g., 7-10% or about 9%); 1-10% (w/w) L-cysteine (e.g., 2-5% or about 3%); 5-15% (w/w) glycine (e.g., 7-10% or about 9%); 1-10% (w/w) L-histidine (e.g., 3-5% or about 4%); 0% L-isoleucine; 0% L-leucine; 5-15% (w/w) L-lysine (e.g., 7-10% or about 8%); 0.5-10% (w/w) L-methionine (e.g., 1-3% or about 2%); 1-10% (w/w) L-phenylalanine (e.g., 4-7% or about 6%); 5-15% (w/w) L-proline (e.g., 6-10% or about 7%); 1-10% (w/w) L-serine (e.g., 4-7% or about 5%); 1-10% (w/w) L-threonine (e.g., 5-8% or about 7%); 0.5-10% (w/w) L-tryptophan (e.g., 1-4% or about 3%); 1-10% (w/w) L-tyrosine (e.g., 7-10% or about 6%); 0% L-valine; 0.02-5% (w/w) L-carnitine (e.g., 0.05-0.25% or about 0.2%); and 0.02-5% (w/w) taurine (e.g., 0.2-0.5% or about 0.3%). An example of a suitable amino acid blend for MSUD is shown in Table 2 in the Examples section.

The blend of amino acids can comprise can comprise from about 10% to about 60% amino acids by weight of the medical food emulsions. “About” indicates that the percentage by weight can vary by up to 2% above or below the recited value. Thus, a percentage of about 10% by weight can include, for example, 8% by weight, 8.5% by weight, 9% by weight, 9.5% by weight, 10.0% by weight, 10.5% by weight, 11% by weight, 11.5% by weight or 12% by weight. A percentage of about 60% by weight can include, for example, 58% by weight, 58.5% by weight, 59% by weight, 59.5% by weight, 60.0% by weight, 60.5% by weight, 61% by weight, 61.5% by weight or 62% by weight. The exact percentage can vary depending upon the particular formulation of medical food emulsion. Useful percentages by weight can include about 10%, about 12%, about 15%, about 20%, about 25% or about 30%.

The amino acids can be in the form of a mixture of single amino acid residues. In some embodiments, at least some of the amino acids can be joined to one another. The amino acids can be joined via a peptide bond, i.e., a chemical bond formed between the carboxyl group of one amino acid and the amino group of another amino acid. Alternatively, or in addition, some of the amino acids can be joined to one another to form di-peptides, tri-peptides, or peptides of other lengths. In some embodiments, the peptides can be fragments of full-length naturally occurring or synthetic proteins. The amino acid residue excluded from the amino acid blend can be leucine, isoleucine, valine, or phenylalanine.

The medical food emulsions include a low pH solvent, e.g., an acid. An acid is a molecule that acts as a proton donor and thus increases the H⁺ concentration of a solution. Acids that readily give up protons to water are strong acids, while those with only a slight tendency to give up protons are weak acids. A useful index of the H⁺ ion concentration in a solution is the pH scale; an aqueous solution with a pH of less than 7 is considered to be acidic. Thus, the amino acids can be solubilized in any aqueous solution having a pH below 7.0, e.g., 6.9, 6.5, 6.2, 6.0, 5.5, 5.0, 4.5, 4.0, 3.5, 3.0, 2.8, 2.6, 2.4, 2.2, 2.0, 1.8, 1.6, 1.5, 1.4, 1.3, 1.2, 1.0 and below. A suitable pH is below about 4.0. “About” indicates that the pH can vary by up to 0.2 pH units above or below the recited value. Thus, a pH of “about” 4.0, can include, for example, pH 3.8, 3.85, 3.90, 3.95, 4.0, 4.05, 4.10, 4.15, or 4.20. Useful pH's for solubilization can be about 0.8, 1.0, 1.5, 2.0, 2.5, 2.8, 3.0, 3.2, 3.5, 3.8, 4.0, or 4.2, 4.5, 5.0, 5.2, 5.5 or 5.7.

The specific concentration of acid will depend in part, upon the relative strength of the acid, with stronger acids requiring lower concentrations and weaker acids requiring higher concentrations. Thus, the concentration and lower pH limit for incubation may vary from acid to acid. Appropriate concentrations and pH's are those that result in efficient solubilization of the amino acid formulations while at the same time effectively masking the taste of the amino acids in a manner that does not result in an excessively sour taste in the final product. Thus the acid can comprise from about 5% to about 50% of the medical food emulsion by weight. “About” indicates that the percentage by weight can vary by up to 2% above or below the recited value. Thus, a percentage of about 5% by weight can include, for example, 3% by weight, 3.5% by weight, 4% by weight, 4.5% by weight, 5.0% by weight, 5.5% by weight, 6% by weight, 6.5% by weight or 7% by weight. A percentage of about 50% by weight can include, for example, 48% by weight, 48.5% by weight, 49% by weight, 49.5% by weight, 50.0% by weight, 50.5% by weight, 51% by weight, 51.5% by weight or 52% by weight. The exact percentage can vary depending upon the particular formulation of medical food emulsion. Useful percentages by weight can include about 10%, about 12%, about 15%, about 17%, about 20%, about 22%, about 25%, about 28% or about 30%. Useful percentages are those that result in a medical food with a final pH of about 3.0 to about 5.5. “About” indicates that the pH can vary by up to 0.2 pH units above or below the recited value. Thus, useful final pH's of the medical food emulsions can be about 2.8, 3.0, 3.2, 3.3, 3.4, 3.6, 3.8, 4.0, 4.2, 4.6, 4.8, 4.9, 5.0, 5.2, 5.4, or 5.6.

The acid can be, for example, an organic acid. Examples of useful organic acids include acetic acid, citric acid, lactic acid, adipic acid, malic acid, and tartartic acid. Any combination of two or more acids can also be used. The organic acid can be contained within a vinegar. The vinegar can be made from the oxidation of ethanol in wine, cider, beer, fermented fruit juice, or nearly any other liquid containing alcohol. Vinegar is a liquid produced from the fermentation of ethanol in a process that yields its key ingredient, acetic acid. The acetic acid concentration ranges typically from about 4 to 8 percent by volume up to about 18% by volume. Typical table vinegars are about 5% acetic acid by volume. Natural vinegars can also contain smaller amounts of tartaric acid, citric acid, and other acids.

Any form of vinegar know to those of skill in the art can be used in the medical food emulsion. Varieties of vinegar include, for example, without limitation, white vinegar, made from oxidizing distilled alcohol; malt vinegar, made from ale that is brewed from malted barley; wine vinegar, made from red, white or fortified wines such as sherry; apple cider vinegar, also known as cider vinegar, made from cider or apple must; fruit vinegars, made from fruit wines, e.g., black currant, raspberry, quince, and tomato; balsamic vinegar, made from concentrated juice, or must, of white grapes and then aged in a successive number of casks made of various types of wood (including oak, mulberry, chestnut, cherry, juniper, ash, and acacia); rice vinegar, made from rice wine; coconut vinegar, made from the sap, or “toddy,” of the coconut palm; cane vinegar, made from sugar cane juice; raisin vinegar; date vinegar; beer vinegar; honey vinegar; Chinese black vinegar, an aged product made from rice, wheat, millet, or sorghum, or a combination thereof. The vinegar may also include one or more flavorings such as herbs, e.g., thyme, oregano, or tarragon; natural or artificial fruit flavorings; and spices, e.g., ginger, cloves, chiles

A useful vinegar for the medical food emulsions described herein is balsamic vinegar. Balsamic vinegar is a grape-based product, very dark brown in color with a rich slightly sweet flavor. Some balsamic vinegars may be aged from three to twelve years; other balsamic vinegars are made with red wine vinegar or concentrated grape juice mixed with a strong vinegar which is laced with caramel and sugar.

The medical food emulsions can also include L-carnitine, a quaternary ammonium compound synthesized from the amino acids lysine and methionine. L-carnitine fortification is essential in the nutritional support of leucine catabolic disorders e.g., isovaleric academia; organic acid metabolic disorders, e.g., glutaric aciduria, propionic acidemia and methymalonicacidemia; and urea cycle disorders. The medical food emulsions can also include taurine or 2-aminoethanesulfonic acid, an organic acid that is a derivative of the sulphur-containing (sulfhydryl) amino acid, cysteine.

The medical food emulsions can also include vitamins and minerals. Vitamins are nutrients required in tiny amounts for essential metabolic reactions in the body. Vitamins can act both as catalysts and substrates in chemical reactions. Vitamins are classified as either water-soluble, meaning that they dissolve easily in water, or fat-soluble, and are absorbed through the intestinal tract with the help of lipids. Humans typically require thirteen vitamins; these include the fat-soluble vitamins (vitamin A (retinol, retinoids and carotenoids); vitamin D (ergocalciferol); vitamin E (tocopherol and cholecalciferol; and vitamin K (naphthoquinone)), and the water-soluble vitamins (vitamin B1 (thiamine); vitamin B2 (riboflavin); vitamin B3 (niacin); vitamin B5 (pantothenic acid); vitamin B6 (pyridoxine); vitamin B7 (biotin); vitamin B9 (folic acid); vitamin B12 (cyanocobalamin); vitamin C (ascorbic acid)). A vitamin can also be inositol, (of which the most prominent naturally-ocurring form is myo-inositol, cis-1,2,3,5-trans-4,6-cyclohexanehexyl), a carbocyclic polyol.

Dietary minerals are chemical elements other than carbon, hydrogen, nitrogen, and oxygen that are required to sustain the health of living organisms. In humans, dietary minerals can include calcium, magnesium, phosphorus, potassium, sodium, and sulfur, as well as those minerals that are needed in relatively small quantities and may be referred to as trace elements, for example, chromium, cobalt, copper, fluorine, iodine, iron, manganese, molybdenum, selenium, and zinc.

The amounts of specific vitamins and minerals in the medical food emulsions may be determined by one of skill in the art. The medical food emulsions can include any combination of the vitamins and minerals that is useful in providing appropriate nutrition to the patient. The vitamins and minerals may be used in the form of a mixture or formulation. For example, a formulation of vitamins and minerals can include 10-20% to (w/w) Vitamin A; 0.05-5% (w/w) Vitamin C; 5-10% (w/w) Vitamin D; 0.05-5% (w/w) Vitamin E; 0.02-5% (w/w) Vitamin K; 0.001-1% (w/w) Thiamin; 0.001-1% (w/w) Riboflavin; 0.05-5% (w/w) Niacin; 0.001-1% (w/w) Vitamin B6; 5-10% (w/w) Folate; 0.001-1% (w/w) Vitamin B12; 0.001-1% (w/w) Pantothenic acid; 0.05-5% (w/w) Biotin; 5-10% (w/w) Choline; 5-15% (w/w) Calcium; 0.05-5% (w/w) Chromium; 5-10% (w/w) Copper; 0.05-5% (w/w) Iodine; 0.05-5% (w/w) Iron; 0.05-5% (w/w) Magnesium; 0.001-1% (w/w) Manganese; 0.05-5% (w/w) Molybdenum; 5-15% (w/w) Phosphorus; 0.05-5% (w/w) Selenium; 0.05-5% (w/w) Zinc; 5-15% (w/w); Potassium; 0% Sodium; 0% Chloride; and 0.05-5% (w/w) Inositol. An example of a suitable list of vitamins and minerals is provided in Example 1, Table 3. The mixture can comprise about 1-5% of the total medical food emulsion by weight; for example about 1%, about 2%, about 4%, about 5%.

The medical food emulsions can also include a lipid i.e., any of a heterogeneous group of aliphatic hydrocarbons that are water insoluble and extractable by nonpolar (or fat) solvents. Lipids may be considered to include fatty acids, neutral fats, waxes and steroids. Useful dietary lipids include vegetable oils, i.e., edible fats that are liquid at room temperature and obtained from plant sources. Examples of vegetable oils include, without limitation, soybean oil, sunflower oil, canola oil, low erucic rapeseed oil, cottonseed oil, corn oil, olive oil, or a combination thereof. The amounts of specific oils in the medical food emulsions may be determined by one of skill in the art. The medical food emulsions can include any combination of oil that is useful in providing appropriate nutrition to the patient, promotes amino acid stability and insures a food product of pleasant taste and texture.

The oil or oils can comprise about 3-60% of the total medical food emulsion by weight. “About” indicates that the percentage by weight can vary by up to 2% above or below the recited value. Thus, a percentage of about 3% by weight can include, for example, 1% by weight, 1.5% by weight, 2% by weight, 2.5% by weight, 3.0% by weight, 3.5% by weight, or 4% by weight, 4.5% by weight or 5% by weight. A percentage of about 60% by weight can include, for example, 58% by weight, 58.5% by weight, 59% by weight, 59.5% by weight, 60.0% by weight, 60.5% by weight, 61% by weight, 61.5% by weight or 62% by weight. The exact percentage can vary depending upon the particular formulation of medical food emulsion. Useful percentages by of oil by weight can include about 10%, about 12%, about 15%, about 20%, about 25%, about 30%, about 35%, about 38%, about 40%, about 42%, about 45%, about 48%, about 50%, about 55%, about 58%, or about 60%.

The medical food emulsions can also include one or more carbohydrates. Carbohydrates or saccharides are are straight-chain aldehydes or ketones with many hydroxyl groups added, usually one on each carbon atom that is not part of the aldehyde or ketone functional group. The basic carbohydrate units are monosaccharides, for example, glucose, galactose, and fructose. The general chemical formula of an unmodified monosaccharide is (CH2O)n, where n is any number of three or greater. Monosaccharides can be linked together in almost limitless ways. Two joined monosaccharides are called disaccharides, such as sucrose and lactose. In general, carbohydrates containing between about three to six monosaccharide units are termed oligosaccharides; larger carbohydrates are termed polysaccharides. Polysaccharides, for example, starch, glycogen, or cellulose, can reach many thousands of units in length. Many carbohydrates contain one or more modified monosaccharide units that have had one or more groups replaced or removed.

Useful dietary carbohydrates include, without limitation, sucrose, glucose (dextrose), fructose, honey, starches, including a non-modified starch, a modified starch, an instant starch, a cook-up starch, or a combination thereof, cornstarch, waxy cornstarch, potato starch, rice starch, tapioca, or wheat starch or a combination thereof. The amounts of specific carbohydrates in the medical food emulsions may be determined by one of skill in the art. The medical food emulsions can include any combination of carbohydrates that is useful in providing appropriate nutrition to the patient and insures a food product of pleasant taste and texture.

The carbohydrates can comprise about 1-50% of the total medical food emulsion by weight. “About” indicates that the percentage by weight can vary by up to 0.5% above or below the recited value. Thus, a percentage of about 1% by weight can include, for example, 0.5% by weight, 1.0% by weight, or 1.5% by weight. A percentage of about 50% by weight can include, for example, 49.5% by weight, 50.0% by weight, or 50.5% by weight. The exact percentage can vary depending upon the particular formulation of medical food emulsion. Useful percentages by of carbohydrates by weight can include about 1%, about 2%, about 3%, about 5%, about 7%, about 10%, about 15%, about 20%, about 25%, about 30%, about 35%, about 40%, about 45%, or about 50%.

The medical food emulsions may include one or more other agents useful for example, in promoting the stability of the products or promoting a pleasing taste or appearance of the food product. These other agents can include antioxidants, coloring agents, stabilizers, preservatives, flavoring agents or emulsifiers. Antioxidants and preservative can include for example, ascorbic acid, propyl gallate, tocopherols, butylated hydroxyanisole and butylated hydroxytoluene. Stabilizers can include thickeners and gelling agents, e.g., agar or pectin. Food colorings can be natural or chemically synthesized compounds. Flavoring agents can be natural or or chemically synthesized compounds, e.g., the essential oil, oleoresin, essence or extractive, protein hydrolysate, distillate, or any product of roasting, heating or enzymolysis, which contains the flavoring constituents derived from a spice, fruit or fruit juice, vegetable or vegetable juice, edible yeast, herb, bark, bud, root, leaf or any other edible portions of a plant, meat, seafood, poultry, eggs, dairy products, or fermentation products thereof, whose primary function in food is flavoring rather than nutritional.

The medical food emulsions can also include flavorings and additives from natural sources including herbs, e.g., including, without limitation, Italian seasoning, marjoram, thyme, rosemary, savory, sage, oregano basil, dill, bay leaf, parsley, chives; spices, e.g., paprika, garlic powder, onion powder, chile powder, pepper, cumin, cinnamon, nutmeg, allspice, mace; condiments, e.g., table salt, kosher salt, lemon juice, mustard, mayonnaise or mayonnaise-like products, onions, garlic, chutney, horseradish, cocktail sauce, relish, ketchup, salsa, soy sauce, tartar sauce, tomato sauce, chipotle chiles, wasabi, worcester sauce, tabasco sauce or non-dairy creamer. Any appropriate additive may be used in the formulation of a medical food emulsion, provided that the additive lacks or substantially lacks the amino acid or acids that are detrimental to the patient having the particular IEPM for which the medical food emulsion is intended. Such additives may be prepared by the practitioner using standard methods and ingredients known in the art. Alternatively or in addition, the additives may be purchased from commercial sources. For example, mayonnaise-like products can include Miracle Whip™ (Kraft Foods) and a non-dairy creamer can include Coffee-mate™ (Nestle).

An emulsifier (also known as an emulgent or surfactant) can be any substance which stabilizes an emulsion. Examples of food emulsifiers include, without limitation, monoglycerides, acetylated monoglycerides, lactylated monoglyceride, polyglycerol esters of fatty acids.

The medical food emulsions can be formulated in a variety of ways to meet the nutritional needs of patients with IEPM. For example, medical food emulsions can be formulated as a salad dressing, dipping sauce, condiment or garnish for use with cooked or uncooked vegetables or other ingredients that are within the dietary restrictions of the particular metabolic disorder of the patient. The precise formulation of the salad dressing, dipping sauce, condiment or garnish can vary according to the nature of the ingredients added to the emulsion. For example, a salad dressing can include at least one of an oil, natural flavorings such as herbs, spices or condiments, emulsifiers, carbohydrates and water. More specifically, a salad dressing can include balsamic vinegar, water, mustard, honey, oil, herbs, an emulsifier, vitamins, minerals, carnitine, inositol and amino acids, wherein the amino acids are exclusive of at least one naturally occurring amino acid residue. Other medical food emulsions can include an enchilada sauce, a vitamin and/or mineral, an organic acid, water, and a blend of amino acids suitable for administration to a patient having an inborn error in protein metabolism.

Methods of Manufacture

Also provided are methods of making a medical food emulsion. The medical food emulsion can be prepared by solubilizing one or more amino acids, exclusive of at least one naturally occurring amino acid, in a low pH solvent to form a homogeneous suspension. The amino acids can be can be solubilized in any low pH solution suitable for human consumption that does not result in amino acid hydrolysis and that maintains the amino acids as a homogeneous suspension. In general, a homogeneous suspension of amino acids is one in which the amino acids are uniformly distributed in a liquid medium and that does not contain visible particulates.

The solvent may be subjected to mild or moderately elevated temperatures relative to ambient temperature. Mildly elevated temperatures can include temperatures ranging from about 28° C. to about 44° C., e.g., 28° C., 29° C., 30° C., 31° C., 32° C., 33° C., 34° C., 35° C., 36° C., 37° C., 38° C., 39° C., 40° C., 41° C., 42° C., 43° C. or 44° C. Moderately elevated temperatures can include temperatures ranging from about 40° C. to about 98° C., e.g., 40° C., 45° C., 50° C., 55° C., 60° C., 65° C., 70° C., 75° C., 80° C., 85° C., 90° C., 95° C., or 98° C. The solvent can be subjected to elevated temperatures either before the addition of the amino acids, at the same time as the addition of the amino acids, or after the addition of the amino acids. “About” indicates that the temperature can vary by up to 2° C. above or below the recited value. Thus, a temperature of “about” 30° C. can include, for example, 28.0° C., 28.5° C., 29.0° C., 2.95° C., 30.0° C., 30.5° C., 31.0° C., 31.5° C., or 32.0° C. The temperature may be measured either in degrees Celsius or degrees Fahrenheit. One of ordinary skill in the art will be able to convert temperatures expressed in degrees Celsius to the corresponding temperatures in degrees Fahrenheit; and conversely, to convert temperatures expressed in degrees Fahrenheit to the corresponding temperatures in degrees Celsius. Useful temperatures can be for example, 40° C., 60° C., or 70° F.

The solvent and the amino acid mixture can be physically agitated by any means known to those in the art. For example, the mixture may be blended, stirred, shaken using standard food preparation equipment. The length of mixing time will vary according to the batch size, mixing apparatus and temperature of the mixture, but should be sufficient to produce a homogeneous suspension. For example, the mixing time can be from about 0.5 minutes to about 10 minutes. The mixing time can be, for example, for about 0.5, 1.0, 1.5, 2.0, 2.5, 3.0, 3.5, 4.0, 4.5, 5.0, 5.5, 6.0, 7.0, 8.0, 9.0, 10.0 or more minutes. “About” indicates that the swelling time can vary by up to 0.2 minutes above or below the recited value. Thus, a swelling time of “about” 2 minutes can include, for example, 1.8 minutes, 1.85 minutes, 1.90 minutes, 1.95 minutes, 2.0 minutes, 2.05 minutes, 2.10 minutes, 2.15 minutes, or 2.20 minutes. Useful mixing times can be 0.5 minutes, 1.0 minutes, 2.5 minutes, 3.0 minutes, 3.5 minutes, 4.0 minutes, 4.5 minutes, 5.0 minutes.

In some embodiments, additional ingredients can be added to the solvent and amino acid mixture following solubilization. The additional ingredients may be added sequentially or concurrently. Any mixing conditions that maintain the amino acids as a homogeneous suspension, i.e., the amino acids are uniformly distributed in the liquid medium and that does not contain visible particulates. The resulting mixture may be physically agitated to combine the additional ingredients with the amino acid mixture, using any of the methods described above. The physical agitation step may be performed at room temperature or at mild to moderately elevated temperatures relative to room temperature, as described for the solubilization step.

Optionally, the medical food emulsions can be submitted to treatments to diminish the bioburden. A process used to inactivate or kill “substantially all” microorganisms (e.g., bacteria, fungi (including yeasts), and/or viruses) in the medical food emulsions is a process that reduces the level of microorganisms in the medical food emulsions by least 10-fold (e.g., at least: 100-fold; 1,000-fold; 10⁴-fold; 10⁵-fold; 10⁶-fold; 10⁷-fold; 10⁸-fold; 10⁹-fold; or even 10¹⁰-fold) compared to the level in the medical food emulsions prior to the process. Suitable conditions for sterilization are hot fill sterilization at 106° C. for 30 seconds in oxygen barrier packaging. Any standard assay method may be used to determine if the process was successful. These assays can include techniques that directly measure microbial growth, e.g., the culture of swab samples on artificial growth media, or molecular detection methods, such as quantitative PCR.

Applications

The medical food emulsions can be administered to patients having inborn errors of protein metabolism or who are at risk for inborn errors of protein metabolism. Particular IEPM's include phenylketonuria, maple syrup urine disease, tyrosinemia, isovaleric acidemia, homocystinuria, organic acid metabolic disorder, and urea cycle disorders.

Phenylketonuria (PKU)

The primary metabolic defect in PKU is the inability to convert excess dietary phenylalanine to tyrosine. As a result, phenylalanine accumulates in the blood and cerebrospinal fluid (CSF) and is excreted in excess in the urine. Abnormally high levels of phenylalanine are diverted to the formation of phenylpyruvic acid and its metabolic derivatives, phenylacetic, phenyllactic acid and orthohydroxyphenylacetic acids. These acids are also excreted in excess. In addition, tyrosine and tryptophan metabolism is abnormal and unusual intermediary products of these two amino acids appear in the urine.

Mental retardation, which can be severe, can result when PKU is left untreated. Petit and grand mal seizures occur frequently, and there also is a high incidence of abnormal electroencephalograms, even in the absence of convulsions. The neurologic manifestations in untreated patients include muscular hypertonicity, exaggerated tendon reflexes, tremors and hyperkinesis. In about 15-20% of the untreated cases, a dermatitis resembling infantile eczema is reported. Many cases demonstrate disorders of pigment metabolism. The earlier PKU is detected and treated, the better an individual's prognosis.

Nutritional support is used to limit the intake of phenylalanine. However, a certain minimum amount of phenylalanine must be consumed to facilitate normal growth and tissue repair. The symptoms of insufficient phenylalanine intake include apathy, anorexia, hypoglycemia, and vacuolization of the marrow erythroid and myeloid cytoplasm. The phenylalanine requirement in terms of body weight decreases rapidly during the first year of life, and frequent assessment and readjustment are necessary in that time.

All naturally occurring proteins contain approximately the same amount of phenylalanine (about 4-6% of total amino acids). Thus, it is not possible to provide enough protein for the growing child without exceeding the desired restriction on phenylalanine. One approach has been to supply a mixture of synthetic L-amino acids that contains no phenylalanine. Tyrosine is an essential amino acid and since it is the distal metabolic product of phenylalanine conversion it is necessary in such dietary formulations to include sufficient tyrosine to meet nutritional requirements. Several medical foods have been developed for the nutritional support of patients with PKU.

Tyrosinemia

Tyrosinemia Type 1 is an inherited disorder of tyrosine metabolism that is caused by deficient fumarylacetoacetate hydrolase activity. Patients present with severe liver and renal disease in infancy and in later childhood develop hepatomas. Biochemically, the disease is characterized by high plasma levels of tyrosine and methionine and increased excretion of tyrosine metabolites.

A diet low in tyrosine, phenylalanine, and often methionine has until recently been the only treatment for type 1 tyrosinemia. Such treatment does not appear to prevent the long term development of hepatic complications, and attention has focused on the recent success of liver transplantation. The diet must be administered until a matching liver for transplantation is located.

Tyrosinemia Type II is associated with autosomal recessive inheritance and has distinctive metabolic abnormalities, including increased levels of tyrosine in the plasma and urine, and increased levels of tyrosine metabolites in the urine. The defect in oculocutaneous tyrosinemia is in the tyrosine aminotransferase of the hepatic cytosol, an enzyme that normally catalyzes the conversion of tyrosine to p-hydroxyphenylpyruvic acid. Deficient enzyme activity results in tyrosine accumulation and blood tyrosine concentrations become elevated. This syndrome is often associated with a characteristic clinical syndrome of eye and skin lesions, permanent neurological damage, mental retardation, and blindness. Early diagnosis is of paramount importance. Treatment typically consists of a low-tyrosine, low-phenylalanine diet.

The symptoms of a related condition, alcaptonuria, first appear in adult life in the form of a discoloration in the connective tissue (ochronosis) and a characteristic arthritis transmitted as an autosomal recessive absence of homogentisic oxidase. This results in excretion of homogentisic acid in the urine.

Maple Syrup Urine Disease (MSUD)

MS UD is another inborn error of metabolism that the present compositions are designed to treat. Infants with this condition appear normal at birth, but soon develop a poor appetite, become apathetic and lethargic, and then manifest neurologic signs. Alternating periods of atonia and hypertonicity appear, followed by convulsions and respiratory irregularities. MSUD was so-named because of the accompanying characteristic odor in the urine, perspiration and ear wax. If this disease is left untreated, it is almost always fatal in the first weeks of life.

The classical form of MSUD involves an almost complete deficiency of branched-chain keto acid dehydrogenase complex. The metabolic event that causes MSUD is a failure of the oxidative decarboxylation of the branched chain amino acids, leucine, isoleucine and valine. As a consequence, the keto acid derivatives accumulate in the blood and are excreted in the urine. There is also an accumulation of the branched chain amino acids. Alloisoleucine also appears, as a result of the enolization of the alpha-keto beta methylvalerate.

MSUD can be treated with a diet providing a limited intake of the branched chain amino acids. Following nutritional support as indicated, the characteristic MSUD odor disappears, neurologic manifestations gradually improve, the electroencephalogram returns to normal, and the abnormal plasma accumulation of the branched chain amino acids and their keto acid derivatives decreases.

Initial treatment normally requires the use of a diet completely devoid of leucine, isoleucine, and valine. Supplementation is begun as the plasma levels of these amino acids approach normal values. Usually, valine and isoleucine become normal several days before the leucine level is in the normal range. After biochemical control is achieved, the intake of the branched chain amino acids can be provided in the form of prescribed amounts of infant formula, milk or low protein foods.

Isovaleric Acidemia

Isovaleric acidemia is an inherited defect of leucine metabolism characterized by the presence of high levels of isovaleric acid (IVA) in the blood and urine. IVA is a short chain fatty acid whose only known amino acid precursor is leucine. In this condition, the activity of isovaleryl CoA-dehydrogenase, the enzyme for IVA degradation, is deficient and is the enzyme defect in this disorder. The metabolic block is the failure to convert isovaleryl-CoA to beta-methylcrotonyl-CoA, resulting in the large accumulation of IVA and metabolites, isovalerylglycine and beta-hydroxyisovaleric acid in the blood and urine. Even in remission these metabolites are present in increased quantities.

Since IVA seems to be solely derived from leucine, which is an essential amino acid, reduction in dietary leucine is effective in controlling the abnormal accumulation of metabolites as well as sequelae. Further, since glycine conjugation with isovaleryl-CoA dehydrogenase is instrumental in preventing IVA accumulation, it is advisable to restrict substances which compete for glycine conjugation, such as benzoic and salicylic acids. The administration of glycine favors the formation of non-toxic isovalerylglycine (IVG) from precursor IVA and hence the consequent diminution of toxic levels of IVA in blood and tissue. Glycine therapy is particularly beneficial for the treatment of acute ketoacidotic episodes in older infants and children, and for the management of acute neonatal disease. Glycine markedly reduces the rise in serum IVA produced by a leucine load. Glycine administration is associated with a pronounced increase in excretion of IVG and in hippurate excretion; both IVG and hippurate excretion being increased significantly by glycine administration as compared with the administration of leucine alone.

In isovaleric acidemia, when adequate carnitine is available, a new metabolite, isovalerylcarnitine (WC), is excreted in large amounts. The use of carnitine therapy in isovaleric acidemia appears to be as effective as glycine therapy in the removal of isovaleryl-CoA and is more effective in reducing plasma IVA. IVC formation is not enhanced by glycine supplementation, and renal loss does not appear to account for the initially diminished levels of free carnitine.

Homocystinuria

The basic metabolic defect in homocystinuria is a deficiency in the activity of the enzyme cystathionine synthetase, which catalyzes an essential step in the trans-sulferation pathway associated with cysteine synthesis. Typically, this enzyme deficiency results in abnormal levels of homocysteine in the urine. About half of the diagnosed cases of homocystinuria are associated with mental retardation. Other clinical symptoms include: ectopia lentis (dislocated lenses) and a number of skeletal deformities. Arterial and venous thromboses are frequent occurrences and are responsible for sudden death. These effects are secondary to the damage caused to the blood vessel walls by homocystiene. Therapy should be attempted in all cases of homocystinuria in an effort to avoid the serious consequences described above.

There are at least two types of homocystinuria caused by cystathione synthetase deficiency. One form is amenable to therapy with large doses of pyroxidine. The other form requires a diet restricted in methionine and supplemented with cystiene. Both biochemical and clinical responses have been reported with diets low in methionine. These diets must be supplemented with cystiene, since the site of the metabolic block makes cystiene a dietary essential for these individuals.

Organic Acid Metabolic Disorders

The disorders of propionate metabolism, methylmalonic acidemia (MMA) and propionic acidemia (PA), are the most common disorders of organic acid metabolism. These disorders usually present in the neonatal period or early infancy with vomiting, lethargy and metabolic acidosis, which may progress to coma and death. The mainstay of treatment of PA and MMA is a diet restricted in isoleucine, methionine, threonine and valine. An inadequate isoleucine, methionine, threonine and valine intake leads to poor growth with chronic malnutrition, a serious complication of the organic acidemias.

Another organic acid metabolic disorder is Glutaric Aciduria Type 1 (GA-1). GA-1 is caused by a deficiency in the activity of glutaryl-CoA dehydrogenase resulting in an accumulation of glutaryl-CoA and its hydrolysis product glutaric acid, which is toxic. Glutaryl-coA is an intermediate in the catabolic pathways of lysine, hydroxylysine and tryptophan. Patients typically present with elevated plasma and urine concentrations of L-glutaric acid and there is a marked increase in the concentrations of tactic acid, isobutyric acid, isovaleric acid and alpha-methylbutyric acid in the urine.

Therapeutic approaches for the control of GA-1 include long term nutritional support using diets low in tryptophan and lysine. Riboflavin, a co-enzyme for glutaryl CoA dehydrogenase, has been given to enhance residual enzyme activity. Large doses of L-carnitine has been used to stimulate excretion of short chain acylcarnitine derivatives of glutaric acid.

Urea Cycle Disorders

Interruptions in the metabolic pathway for urea synthesis are caused by the deficiency or inactivity of any one of several enzymes involved in specific steps in the cascade. The common pathologic sequalae of these clinical disorders is the extreme elevation of the plasma ammonia level. Typically associated with this increase in ammonia buildup are acute episodes of vomiting, lethargy, convulsions and abnormal liver enzyme levels. Protracted exposure to high levels of plasma ammonia leads to mental and physical retardation. If left untreated prolonged exposure to high levels of plasma ammonia is fatal typically following a period of lethargy, convulsions and coma.

Several enzyme deficiencies have been noted as contributing to urea cycle disorders. These include: N-acetyl glutamate synthetase deficiency, which causes neurologic deterioration due to elevated blood ammonia; carbamyl phosphate synthetase (CPS) deficiency which is often a lethal disease with death occurring in the first weeks of to life; ornithine transcarbamylase deficiency (OTD) which is inherited in a sex-linked dominant manner and is generally fatal in the newborn male; argininosuccinic acid synthetase which typically results in severe neurological impairment leading to mental retardation or death; argininosuccinate lyase deficiencies result in clinical manifestations of retardation, spasticity, and episodes of convulsions; arginase deficiency, which results in severe neurologic deterioration over time.

All of these disorders respond to some degree to restriction of protein intake. Acute episodes are usually precipitated by an increased protein intake, an infection or any incident that leads to a negative nitrogen balance. These acute episodes are best handled by the omission of protein and intravenous fluid therapy. Prolonged treatment of children by limiting protein intake to the minimal requirement together with adequate energy intake and supplements of essential amino acids has resulted in control of the plasma ammonia levels and alleviation of the clinical symptoms. No single panacea is available and nutritional support is specific to the individual disorder. For example, in the case of CPS and OTD deficiencies dietary supplementation with arginine may be effective treatment for reducing plasma ammonia levels. Arginine is considered to be an essential amino acid in this disturbance because of the site of the metabolic block.

The medical food emulsions can be enterally administered to patients as part of their ordinary daily diet. The amount of the medical food emulsions fed to a patient will vary depending upon the nature of the formulation, the patient's condition, the patient's body weight, age, and gender, the total daily dietary intake of particular amino acids and the judgment of the attending clinician. The medical food emulsions may be administered as nutritional support to both adults and children, as well as to provide nutritional support during pregnancy. The efficacy of medical food emulsions can be monitored by standard methods known in the art, for example, analysis of levels of specific amino acids or their metabolites in a patient's blood, serum or urine.

The medical food emulsions can be administered in conjunction with other medical foods or therapeutic modalities to an individual in need of treatment. Other therapeutic modalities will vary according to the particular disorder but can include, for example, cofactor supplementations, such as administration of sodium bicarbonate, carnitine, and vitamin B12, kidney dialysis and liver transplantation; evolving therapies include somatic gene therapy.

Articles of Manufacture

Also disclosed are articles of manufacture that can the medical food emulsions as provided herein. An article of manufacture can include, for example, medical food emulsions. In addition, an article of manufacture further may include, for example, packaging materials and instructions for use.

### EXAMPLES

### Example 1

### Materials and Methods

PKU amino acid mixture. A PKU amino acid formula useful for adults is shown in Table 1.

TABLE 1
PKU Amino Acid Formula

| Component | Content (g) per 100 g |
|---|---|
| L-alanine | 3.95 |
| L-arginine | 7.56 |
| L-aspartic acid | 7.56 |
| L-cysteine | 2.52 |
| glycine | 7.56 |
| L-histidine | 3.87 |
| L-isoleucine | 6.72 |
| L-leucine | 10.92 |
| L-lysine | 7.98 |
| L-methionine | 2.18 |
| L-phenylalanine | 0 |
| L-proline | 7.14 |
| L-serine | 5.04 |
| L-threonine | 6.72 |
| L-tryptophan | 2.18 |
| L-tyrosine | 10.08 |
| L-valine | 7.56 |
| L-carnitine | 0.08 |
| taurine | 0.34 |
| Total amino acids, g | 100 |

MSUD amino acid mixture. A MSUD amino acid formula suitable for adults is shown in Table 2.

TABLE 2
MSUD Amino Acid Formula

| Component | Content (g) per 100 g |
|---|---|
| L-alanine | 10.499 |
| L-arginine | 8.749 |
| L-aspartic acid | 8.749 |
| L-cysteine | 3.062 |
| glycine | 8.749 |
| L-histidine | 4.025 |
| L-isoleucine | 0.0 |
| L-leucine | 0.0 |
| L-lysine | 8.312 |
| L-methionine | 2.2757 |
| L-phenylalanine | 5.687 |
| L-proline | 7.437 |
| L-serine | 5.249 |
| L-threonine | 6.562 |
| L-tryptophan | 2.625 |
| L-tyrosine | 6.124 |
| L-valine | 0.0 |
| L-carnitine | 0.175 |
| taurine | 0.35 |
| Total amino acids, g | 100 |

Vitamin/mineral mixture. A Vitamin/mineral mixture suitable for adults is shown in Table 3.

TABLE 3
Vitamins/mineral formulation

| | Content | Content |
|---|---|---|
| | (mg) per | (mg) per |
| Micronutrient | 245 mg | 1 g |
| Vitamin A, IU | 38.4 | 156.73 |
| Vitamin C, mg | 1.27 | 5.18 |
| Vit D, IU | 6.4 | 26.12 |
| Vit E, IU | 0.37 | 1.51 |
| Vit K, mcg | 1.4 | 5.71 |
| Thiamin, mg | 0.02 | 0.08 |
| Riboflavin, mg | 0.02 | 0.08 |
| Niacin, mg | 0.27 | 1.10 |
| Vit B6, mg | 0.03 | 0.12 |
| Folate, mcg | 8.93 | 36.45 |
| Vit B12, mcg | 0.07 | 0.29 |
| Pantothenic acid, mg | 0.08 | 0.33 |
| Biotin, mcg | 0.73 | 2.98 |
| Choline, mg | 5 | 20.41 |
| Calcium, mg | 22 | 89.80 |
| Chromium, mcg | 0.5 | 2.04 |
| Copper, mcg | 14.7 | 60.00 |
| Iodine, mcg | 1.5 | 6.12 |
| Iron, mg | 0.27 | 1.10 |
| Magnesium, mg | 4.53 | 18.49 |
| Manganese, mg | 0.03 | 0.12 |
| Molybdenum, mcg | 0.73 | 2.98 |
| Phosphorus, mg | 19 | 77.55 |
| Selenium, mcg | 1.04 | 4.24 |
| Zinc, mg | 0.18 | 0.73 |
| Potassium, mg | 20 | 81.63 |
| Sodium, mg | 0 | 0.00 |
| Chloride, mg | 0 | 0.00 |
| Inositol, mg | 1 | 4.08 |

### Example 2

### Balsamic Vinaigrette: PKU Formulation

The respective weights of the ingredients and their percentages in the final product are listed in Table 4. The PKU formulation of balsamic vinaigrette was prepared as follows. An aqueous 30% acetic acid solution (i.e., 30% vinegar), pH 2.5, was combined with the PKU amino acid blend described in Example 1 at a temperature of 60° C. The resulting pH was 2.8. The mixture was blended for 120 seconds; the blending speed was reduced and additional ingredients were added in the following order: oils, emulsifiers, water, vitamins, honey, and seasoning. Blending continued for 8 minutes; the temperature of the mixture was maintained at 40-60° C. The emulsion was hot fill sterilized at 106° C. for 30 seconds in oxygen barrier packaging. The percentages of acetic acid and PKU amino acid blend in the final product were 14.52% and 16.58%, respectively, as shown in Table 4.

TABLE 4
Balsamic vinaigrette: PKU formulation

| Ingredients | Percent (w/w) |
|---|---|
| Balsamic Vinegar | 14.52 |
| Water | 10.73 |
| PKU amino acid blend | 16.58 |
| Vitamin/mineral blend | 4.03 |
| Dijon mustard | 8.12 |
| Honey | 7.52 |
| Oil | 37.01 |
| Emulsifier | 1.48 |
| Totals | 100.00 |

### Example 3

### Italian Balsamic Vinaigrette: PKU Formulation

The respective weights of the ingredients and their percentages in the final product are listed in Table 5. The PKU formulation of Italian balsamic vinaigrette was prepared as follows. Balsamic vinegar was heated to 70° F. and then mixed with water. The PKU amino acid mixture, the vitamin/mineral mixture (both as described in Example 1), Italian seasoning, paprika, garlic powder, and onion powder were added and mixed until evenly distributed. The liquid was partially saturated with particulates and became somewhat viscous. The oil was then added in a steady stream while the vinegar mixture was rapidly mixed in a blender. The salt was then added with blending. The final pH of the emulsion was 5.0. A serving of one ounce or 29 grams contained 5 grams of protein equivalent.

TABLE 5
Italian Balsamic Vinaigrette: PKU Formulation

| | Percent |
|---|---|
| Ingredients | (w/w) |
| Balsamic Vinegar | 14.49 |
| Water | 12.03 |
| PKU amino acid mixture | 17.67 |
| Vitamin/mineral mixture | 3.41 |
| Italian Seasoning (blend of marjoram, thyme, | 0.30 |
| rosemary, savory, sage, oregano, and basil) | |
| Paprika | 0.71 |
| Garlic powder | 1.25 |
| Onion powder | 0.85 |
| Salt | 1.19 |
| Oil | 48.10 |
| Totals | 100.00 |

### Example 4

### Enchilada Sauce: PKU Formulation

The respective weights of the ingredients and their percentages in the final product are listed in Tables 6 and 7. The enchilada sauce emulsion was prepared by combining the PKU amino acid mixture, the vitamin/mineral mixture, citric acid and water with an enchilada sauce base as follows. To prepare the enchilada sauce base, the oil was heated in a saute pan on medium heat to a temperature below the smoke point (400° F.) of refined canola oil. When the temperature of the oil reached 300° F., the onions, garlic, and cumin were added. The mixture was stirred, then covered and cooked for one minute, then removed from the heat to avoid scorching the garlic and creating a bitter taste. The remaining enchilada sauce base ingredients were added (i.e., ground chile powder, canned tomato sauce, water, wheat starch, chipotle pepper and kosher salt), and the resulting mixture was stirred and cooked for 2-3 minutes at 300° F. until the liquid was just slightly thickened and reduced in volume. The mixture was removed from the heat and cooled to 150° F. The reduced mixture was weighed and the moisture loss from cooking was calculated. The cooled mixture was transferred to a blender and blended to homogeneity.

The enchilada sauce emulsion was prepared by adding the enchilada sauce base to a mixture of the requisite amounts of the PKU amino acid mixture, the vitamin/mineral mixture, citric acid and water. The ingredients were mixed until thoroughly combined to form an emulsion. In some embodiments, the consistency of the enchilada sauce was adjusted by increasing or decreasing the relative amount of water. The final pH was between 4.8 and 4.9. A serving of one ounce or 29 grams contained 5 grams of protein equivalent.

TABLE 6
Enchilada Sauce Base

| | | Percent |
|---|---|---|
| Ingredients | Batch weight | (w/w) |
| Refined canola oil | 14 | 4.48 |
| Onion, yellow, diced | 39.45 | 12.62 |
| Garlic, minced | 8 | 2.56 |
| Cumin, ground | 2.4 | 0.77 |
| Chili powder, ground | 10.76 | 3.44 |
| Tomato sauce, canned | 96.6 | 30.89 |
| Water | 115.59 | 36.97 |
| Wheat starch | 3.7 | 1.18 |
| Chipotle pepper, canned, | 21.18 | 6.77 |
| rinsed, seeds removed | | |
| Kosher salt | 1 | 0.32 |
| Totals | 312.68 | 100.00 |
| Adjusted total for moisture | 270 | |
| loss | | |

TABLE 7
Enchilada Sauce Emulsion: PKU Formulation

| Ingredients | Batch weight | Percent (w/w) |
|---|---|---|
| Enchilada sauce | 20.19 | 69.72 |
| PKU amino acid mixture | 5.95 | 20.55 |
| Vitamin/mineral mixture | 1.15 | 3.97 |
| Citric acid | 0.22 | 0.76 |
| Water | 1.45 | 5.01 |
| Totals | 28.96 | 100.00 |

### Example 5

### Ranch Dressing: MSUD Formulation

The respective weights of the ingredients and their percentages in the final product are listed in Table 8. The MSUD formulation of ranch dressing was prepared as follows. All ingredients were brought to room temperature, e.g., to about 70° F. The non-dairy creamer and lemon juice were combined and incubated for 10 minutes at room temperature. The MSUD amino acid mixture, the vitamin/mineral mixture, onion powder, garlic powder, parsley, dill, chives, salt and pepper were added to the non-dairy creamer-lemon juice blend and mixed until evenly distributed. The mayonnaise was then added, the resulting mixture blended until smooth and incubated for at least one hour to allow flavors to develop. The pH of the final product was approximately 4.9. A serving of one ounce or 29 grams contained 5 grams of protein equivalent.

TABLE 8
Ranch dressing: MSUD formulation

| | Batch | Percent |
|---|---|---|
| Ingredients | weight | (w/w) |
| Non-dairy creamer | 10.55 | 11.74 |
| Lemon juice | 20.54 | 22.86 |
| MSUD amino acid mixture | 17.15 | 19.09 |
| Vitamin/mineral mixture | 3.45 | 3.84 |
| Onion powder | 0.36 | 0.40 |
| Garlic powder | 0.45 | 0.50 |
| Parsley, dried | 0.06 | 0.07 |
| Dill, dried | 0.03 | 0.03 |
| Chives, dried | 0.06 | 0.07 |
| Salt | 0.45 | 0.50 |
| Black pepper | 0.06 | 0.07 |
| Salad dressing (Miracle-whip type) | 36.7 | 40.84 |
| Totals | 89.86 | 100.00 |

A number of embodiments of the invention have been described. Nevertheless, it will be understood that various modifications may be made without departing from the spirit and scope of the invention. Accordingly, other embodiments are within the scope of the following claims.

1. A medical food emulsion comprising:
(a) a low pH solvent; and
(b) a blend of amino acids suitable for administration to a patient having an inborn error in protein metabolism.

2. The medical food emulsion of claim 1, wherein the low pH solvent comprises about 5% to 60% of the emulsion, and the blend of amino acids (a) comprises about 10% to 60% of the emulsion by weight and (b) excludes at least one naturally occurring amino acid residue.

3. The medical food emulsion of claim 1, wherein the low pH solvent has a pH of about 1.0 to about 3.5 and/or the emulsion has a pH of about 3.0 to about 5.5.

4. The medical food emulsion of claim 1, wherein the low pH solvent comprises an organic acid.

5. The medical food emulsion of claim 4, wherein the organic acid is acetic acid, citric acid, lactic acid, adipic acid, malic acid, tartaric acid or a combination of two or more of these acids.

6. The medical food emulsion of claim 1, wherein the organic acid is contained within a vinegar.

7. The medical food emulsion of claim 1, wherein the amino acids are in the form of a mixture of single amino acid residues.

8. The medical food emulsion of claim 1, wherein at least some of the amino acids are joined to one another.

9. The medical food emulsion of claim 2, wherein the naturally occurring amino acid residue is leucine, isoleucine, valine, or phenylalanine.

10. The medical food emulsion of claim 1, further comprising one or more of:
(a) a carbohydrate;
(b) a lipid;
(c) a vitamin; and
(d) a mineral.

11. The medical food emulsion of claim 10, wherein the carbohydrate comprises a disaccharide.

12. The medical food emulsion of claim 11, wherein the disaccharide is sucrose.

13. The medical food emulsion of claim 10, wherein the carbohydrate comprises a starch.

14. The medical food emulsion of claim 13, wherein the starch is a non-modified starch, a modified starch, an instant starch, a cook-up starch, or a combination thereof.

15. The medical food emulsion of claim 14, wherein the starch is a corn, waxy cornstarch, potato, rice, tapioca, or wheat starch or a combination thereof.

16. The medical food emulsion of claim 10, wherein the lipid comprises a vegetable oil.

17. The medical food emulsion of claim 16, wherein the vegetable oil is soybean oil, sunflower oil, canola oil, low erucic rapeseed oil, cottonseed oil, corn oil, olive oil, or a combination thereof.

18. A medical food emulsion comprising balsamic vinegar, water, mustard, honey, oil, an emulsifier, and a blend of amino acids suitable for administration to a patient having an inborn error in protein metabolism.

19. The medical food emulsion of claim 1, further comprising an antioxidant, a coloring agent, a stabilizer, a preservative, or a flavoring agent.

20. A medical food emulsion comprising a non-dairy creamer, lemon juice, a vitamin and/or mineral, a seasoning and/or spice, a mayonnaise or comparable emulsion, and a blend of amino acids suitable for administration to a patient having an inborn error in protein metabolism.

21. A medical food emulsion comprising an enchilada sauce, a vitamin and/or mineral, an organic acid, water, and a blend of amino acids suitable for administration to a patient having an inborn error in protein metabolism.

22. A medical food emulsion comprising balsamic vinegar, water, a vitamin and/or mineral, a seasoning and/or spice, an oil, and a blend of amino acids suitable for administration to a patient having an inborn error in protein metabolism.

23. The medical food emulsion of claim 1, wherein the emulsion is formulated as a salad dressing or sauce.
